(19) United States
(12) Patent Application Publication
(10) Pub. No.: US 2026/0241052 A1
(43) Pub. Date: Aug. 20, 2026

(54) LIGHTING AND SANITIZING DEVICE

(71) Applicant: NEXTSENSE S.R.L., Salerno (IT) (IT)

(72) Inventors: Rosario Valles, Bellizzi (SA) (IT); Carmelo Raffaele Cartiere, Battipaglia (SA) (IT)

(73) Assignee: NEXTSENSE S.R.L., Salerno (IT) (IT)

(21) Appl. No.: 19/165,455

(22) PCT Filed: Mar. 7, 2024
(86) PCT No.: PCT/IT2024/050049

§ 371 Date: Sep. 15, 2025

(30) Foreign Application Priority Data
Mar. 15, 2023 (IT) 102023000004932

(51) Int. Cl. A61L 2/08; A61L 9/18

(52) U.S. Cl. CPC A61L 2/08; A61L 9/18; A61L 2202/11; A61L 2209/12

(57) ABSTRACT

A lighting and sanitizing device (10, 110, 210, 310) capable of obtaining an effective reduction and/or inhibition of the action of a wide group of microorganisms potentially harmful to people and/or animals. The invention also comprises a lighting apparatus (20) comprising at least one lighting and sanitizing device (10, 110, 210, 310).

### FIELD OF THE INVENTION

The present invention concerns a lighting and sanitizing device capable of reducing and/or inhibiting the action of one or more microorganisms such as, for example, bacteria, viruses, fungi, spores, or molds. The lighting and sanitizing device performs the function of a lighting source that emits visible light with sanitizing effects for both surfaces and the air and therefore for the environment illuminated by it. The lighting and sanitizing device allows a controlled emission and a uniform diffusion of the electromagnetic radiation that allows to obtain an effective reduction and/or inhibition of the action of a wide group of microorganisms potentially harmful to people and/or animals.

### BACKGROUND OF THE INVENTION

Lighting and sanitizing devices employing LED (Light Emitting Diode) technology are known and comprise LED emitters that emit electromagnetic radiations in the visible spectrum and/or in the ultraviolet spectrum, which are used for their bactericidal effect in the environment. However, such known devices have been found to be harmful to humans and do not allow an effective sanitation of the environments and surfaces and are often limited to specific bacterial species and are not effective towards other microorganisms.

It is known that most microbial species, in addition to the ultraviolet radiations, are also sensitive to a part of the wavelengths of the visible light, and in particular to the wavelengths within the range between 400 and 420 nm, comprised in the so-called Soret band.

Application WO2017/179082A1, on behalf of the present Applicant, describes a lighting and sanitizing device comprising three different types of LED emitters, spaced apart from each other, each comprising a green-coloured LED emitter, a red-coloured LED emitter and a blue-violet-coloured LED emitter.

This solution, while allowing to obtain a certain reduction and/or inhibition of the microbial load in the surrounding environment, has the disadvantage that the radiation fluxes emitted by the various groups of LED emitters are not uniformly distributed over the entire emission area of the device, so that at certain points the respective irradiated cones overlap each other while at other points they are separated and non-uniform. Since the irradiated cones are separated, therefore, even by greatly increasing the intensity of energy provided by the electromagnetic radiation it is not possible to obtain an effective removal and/or inhibition of the microorganisms. In addition, their encumbrance does not allow an effective installation inside small-sized lamp holders, or inside other devices with limited dimensions.

Another disadvantage is given by the fact that the presence of the green and red LED emitters shifts the frequency band towards the field of the microwaves, hitting not only the harmful microorganisms, such as the bacteria sensitive to the Soret band, but also other microorganisms that are not as harmful and whose presence is indeed fundamental for a balance between the various species, consequently obtaining a substantially sterile environment. This therefore makes the known solution not very versatile for use in different fields of application. Added to this is the further disadvantage that, although the microbial load break-down times are considerably shorter than other known technologies, they do not lend themselves to applications where a controlled and/or selective sterilization in even shorter times is required.

Solutions are also known relating to a lamp comprising three blue-violet type LED emitters which are separated and spaced from each other, adapted to emit electromagnetic radiations with wavelengths that are close to but different from each other, which are flanked by a white LED emitter adapted to emit a light with continuous spectrum at a certain colour temperature. Even this solution, however, while allowing to obtain a targeted effect on the microorganisms sensitive to the electromagnetic radiations of the Soret band, does not allow to obtain a uniform emission of the radiation, as it is given by the superposition of the radiations emitted by each of the LED emitters. This last known solution also presents the same problems as the previous one.

In general, the known lighting and sanitizing devices, in particular those in which the emission of an effective emission spectrum towards various microbial species is required, have the following problems:
limited precision in emission control, in particular maintaining the electromagnetic radiation in a specific range with an amplitude of less than 20 nm;
problems of uneven distribution of the radiation, causing concentrated or sparse areas of radiation;
problems of interference and energy dispersion between the adjacent LED emitters due to non-optimized mutual distances;
the overall emission of the lighting and sanitizing device is not optimized;
sanitation requires times unsuitable for specific applications;
encumbrances that are not acceptable for specific applications;
reduced effectiveness towards some microbial species and excessive effectiveness towards other microbial species, with consequent lowering of the overall degree of efficiency.

It is also known that in the case of lighting sources that emit electromagnetic radiations at different wavelengths, in which the sources are distinct and separate elements, such as for example in the case of clusters of separate sources or RGBW chips, chromatic aberrations can occur that typically create coloured halos and non-uniform emissions both under the photometric and colorimetric profile, even over the entire volume of the irradiated solid angle.

The need to obtain a compliant, uniform and homogeneous diffusion of electromagnetic radiation is also known, in particular in some fields, including, by way of example, in medicine and biology, in materials sciences, in telecommunications, as well as for the protection of the microbiological contamination of environments and/or materials.

It is also known the need to achieve an effective sanitation both of surfaces and environments in short times with low energy consumption.

There is therefore the need to perfect a lighting and sanitizing device having the function of breaking down, or at least significantly reducing, the microbial load in a surrounding environment that can overcome at least one of the disadvantages of the state of the art.

To do this, it is necessary to solve the technical problem of making available a lighting and sanitizing device suitable for emitting a highly focused beam of electromagnetic radiations and comprising different and uniformly distributed wavelengths which are combined with each other to effectively sanitize environments and/or surfaces without causing harm to people.

One purpose of the present invention is to make available a lighting and sanitizing device capable of overcoming the optical problems typical of the distinct and separate sources underlying a reflector and/or a refractor, which present non-homogeneous emissions having coloured halos that alter the emitted beam with discolorations and non-uniform chromatic renditions on the illuminated objects on all solid emission angles affected by the beam.

Yet another purpose of the invention is to make available a lighting and sanitizing device adapted to emit electromagnetic radiations precisely centred around well-defined values, avoiding undesired effects such as, for example, interference phenomena that reduce the combination of the electromagnetic radiations.

Another purpose of the present invention is to realize a lighting and sanitizing device having a controlled energy emission and a uniform diffusion of the electromagnetic radiation.

Another purpose of the invention is to make available a lighting and sanitizing device that is highly effective in breaking down microorganisms sensitive to the electromagnetic radiations within a well-defined band.

The Applicant has devised, tested and embodied the present invention to overcome the shortcomings of the state of the art and to obtain these and other purposes and advantages.

### SUMMARY OF THE INVENTION

The present invention is set forth and characterized in the independent claim. The dependent claims describe other characteristics of the present invention or variants to the main inventive idea.

In accordance with the above purposes and to resolve the technical problem disclosed above in a new and original way, also achieving considerable advantages compared to the state of the prior art, a lighting and sanitizing device according to the present invention for breaking down the bacterial load, or other microbes, in a surrounding environment comprises an electronic board in which at least one electromagnetic radiation emitter group is installed, wherein said at least one emitter group comprises a triad of primary emitters and five secondary emitters configured to emit an electromagnetic radiation with a longer wavelength than that of all the primary emitters.

Each triad comprises three primary emitters different from each other, each configured to emit an electromagnetic radiation having a wavelength with a specific peak value different from the other two primary emitters.

Here and in the following, by way of example and not for this limiting, the electronic board may be a support package for emitters, for example LEDs. Advantageously, the electronic board can be a package of the type SMD, COB, DOB, Flip LED, PCB, MPCB, or other similar packages.

Preferably, the primary emitters are adapted to emit a light radiation in the violet spectrum, indicatively comprised between 380 nm and 435 nm and the secondary emitters are adapted to emit a light radiation in the blue spectrum, indicatively comprised between 435 nm and 500 nm.

Preferably all the secondary emitters are configured to emit an electromagnetic radiation at a same wavelength.

According to one aspect of the invention, the triad of primary emitters comprises:
a first emitter configured to emit an electromagnetic radiation having a peak value with wavelength comprised between 404 nm and 410 nm, with a preferential peak value of 407 nm;
a second emitter configured to emit an electromagnetic radiation having a peak value with wavelength comprised between 410 nm and 416 nm, with a preferential peak value of 413 nm;
a third emitter configured to emit an electromagnetic radiation having a peak value with wavelength comprised between 418 nm and 424 nm, with a preferential peak value of 421 nm.

The five secondary emitters are configured to emit an electromagnetic radiation having a peak value with wavelength comprised between 452 nm and 458 nm, with a preferential peak value of 455 nm.

Such a combination of primary emitters with different wavelengths together with the secondary emitters in a 1:1:1:5 proportion allows to obtain a substantially uniform overall electromagnetic radiation flux having wavelengths with respective peak values between 400 nm and 470 nm and preferably between 402 nm and 460 nm, even more preferably centred in the range from 404 nm to 424 nm and between 450 nm and 460 nm, making the lighting and sanitizing device particularly suitable for removing microorganisms comprising Gram+, Gram−, viruses, spores, molds, fungi, mites and yeasts that are potentially harmful to humans.

In particular, the superposition of the irradiated cones emitted by the individual emitters with different wavelengths means that the overall cone emitted by the lighting and sanitizing device, defined by the interference and combination of the individual cones emitted by the eight emitters of a group or respective multiples thereof, is already focused and centred on the wavelengths with a high antimicrobial and bactericidal effect.

According to embodiments, the lighting and sanitizing device comprises a number N of emitter groups, for example comprised between 2 and 20, wherein there are altogether N first emitters, N second emitters, N third emitters and 5*N secondary emitters, so that the 1:1:1:5 proportion is maintained and repeated from time to time.

The lighting and sanitizing device effectively combines a plurality of monochromatic sources that can be advantageously encapsulated by a photosensitive coating layer, also called activator layer, so as to obtain the equivalent of a single source capable of emitting visible light with sanitizing effect.

According to embodiments, the lighting and sanitizing device comprises a photosensitive coating layer, which covers the at least one emitter group, i.e. all emitters of a given emitter group are covered by the same photosensitive coating layer.

This photosensitive coating layer has a broad photosensitivity band, comprised between 380 nm and 780 nm, unlike what is provided in the solutions of the prior art that provide coatings with a narrow photosensitivity band, comprised between 460 nm and 470 nm.

Using an electromagnetic radiation generated by the 3 primary emitters, or their multiples, and by the 5 secondary emitters, or their multiples, the photosensitive coating layer allows to obtain a visible light having a wavelength from 400 nm upwards, while maintaining a maximum value centred at the peak values of the three primary emitters. The photosensitive coating layer is de facto configured to absorb the various wavelengths, and go into an excited state to release photons covering a spectrum similar to white light with additionally the peak values of the wavelengths of the primary emitters that are functional to act against the different microorganisms. The photosensitive coating layer is substantially transparent to the wavelengths of the peak values of the primary emitters.

The photosensitive coating layer is configured to modify the wavelength of the emitted radiation, taking it outside the specific wavelength of the respective emitter group, without however substantially modifying the radiation emitted at the wavelength around the Soret band.

This allows to obtain a white light emission centred on the planckian curve having a high colour rendering index (CRI), preferably higher than 80, even more preferably higher than 90 and therefore proximate to natural light, and consequently a controlled and precise energy emission is obtained.

The photosensitive coating layer, in combination with the radiations emitted by the primary and secondary emitters, thus allows a homogeneous light emission to be obtained, substantially devoid of chromatic aberrations, so that the emitted beam is uniform and devoid of discolorations over the entire solid angle of emission.

According to embodiments, the photosensitive coating layer may be based on phosphorus, which may be at least partly doped. When doped, substances can be used which are adapted to provide a red colour, so as to compensate for the blue colour of the radiation emitted at least by the secondary emitters.

According to embodiments, the emitters of the at least one emitter group are distributed on a surface of the electronic board so that the overlapping volume of the respective cones of emitted light comprises the light cones of the primary emitters and at least one of the light cones of the secondary emitters.

According to embodiments, the primary emitters are positioned on the electronic board aligned along at least a first direction, wherein along said at least a first direction there are no first emitters adjacent to each other, second emitters adjacent to each other and third emitters adjacent to each other.

According to embodiments, the distance between primary emitters and/or first close secondary emitters, i.e. side by side, is reduced to a minimum, so as to optimise the combination of the emissions of the individual emitters.

According to embodiments, the distance between the emitters respectively side by side is comprised between 0.001 mm and 1.10 mm, preferably between 0.1 mm and 0.9 mm.

This configuration allows to minimize the interferences and the electrical dispersion between adjacent emitters, and at the same time allows an optimal combination and overlap of the individual cones emitted by each emitter and to obtain a uniform and compliant propagation of the electromagnetic radiations.

In fact, in this way, a single cone of light is emitted from the electronic board, given by the combination of the cones emitted by the individual emitters.

According to embodiments, the primary emitters are more concentrated in a central zone or band, and are at least partly surrounded by the secondary emitters, so as to further optimize emission uniformity.

According to embodiments, the primary emitters and/or the secondary emitters are LED (Light Emitting Diode) emitters.

According to other embodiments, the primary and/or secondary emitters are OLED (Organic Light Emitting Diode) emitters.

According to other possible variants, part of the primary and/or secondary emitters are partly LED and partly OLED emitters.

Preferably the emitters have homogeneous dimensions and shapes, for example rectangular, square, or circular, although different shapes are not excluded.

According to embodiments, each of the primary emitters and of the secondary emitters has a substantially rectangular shape and has respective long sides oriented on parallel longitudinal axes.

According to embodiments, the emitters are preferably arranged aligned in a same direction, or part on a first direction and part on a second direction transverse to the first direction.

In accordance with embodiments, at least the primary emitters are all oriented in the same direction.

In accordance with embodiments, at least in the case where at least two emitter groups are present, all primary and secondary emitters are arranged aligned on parallel axes.

According to embodiments, the first emitters, second emitters and third emitters are aligned on respective transverse rows alternating with rows of secondary emitters.

According to embodiments, the emitters each comprise a chip made of semiconductor material, preferably based on indium gallium nitride, each of which is suitable for emitting a substantially mono-frequency light radiation, i.e. having a desired very focused peak of wavelength.

The lighting and sanitizing device may comprise a plurality of emitters, for example an array of DIE covered by respective photosensitive coating layers, preferably of the high light efficiency type, preferably comprised between 120 and 200 lm/w, even more preferably comprised between 140 and 180 lm/w.

In accordance with embodiments, said primary and secondary emitters within each group G are connected by gold wires, preferably gold having a purity greater than 99%, more preferably greater than 99.95%. The gold connections guarantee excellent electrical conductivity and high resistance to the aggressive chemical agents. In addition, they are not sensitive to volatile chlorine compounds or other substances, so they prevent undesired colourations of the emitters that can occur in the case of silver connections.

The lighting and sanitizing device has been subjected to trials and tests under different conditions to identify the technical characteristics suitable for obtaining a compliant, uniform and homogeneous diffusion of the electromagnetic radiations, among which—by way of example—the following have been identified: that the dimensions of the emitters are proportional, that they are placed at certain mutual distances and that a photosensitive coating layer is applicable on them that acts as a conversion shielding and finally that they generate luminous fluxes at certain values and comprised within certain ranges.

### DESCRIPTION OF THE DRAWINGS

These and other aspects, characteristics and advantages of the present invention will become apparent from the following description of some embodiments, given as a non-restrictive example with reference to the attached drawings wherein:

FIG. 1 is a schematic view of a lighting and sanitizing device in accordance with a first variant of the invention, in which the different emitters are indicated by respective letters “A”, “B”, “C”, “X”;

FIG. 2 is a schematic view of a lighting and sanitizing device in accordance with a second variant of the invention;

FIG. 3 is a schematic view of a lighting and sanitizing device in accordance with a third variant of the invention;

FIG. 4 is a schematic view of a lighting and sanitizing device in accordance with a fourth variant of the invention;

FIG. 5 is a graph showing the emission spectrum of the individual primary emitters and the spectrum resulting from their combination;

FIG. 6 is a graph showing the emission spectrum of the primary and respectively secondary emitters of one or more emitter groups of a lighting and sanitizing device according to the invention respectively alone and in combination with a photosensitive coating layer;

FIG. 7 is a graph showing the emission spectrum of a lighting and sanitizing device according to the invention given by the combination of the radiations emitted by the emitter groups with a photosensitive coating layer;

FIG. 8 is a graph showing the emission spectrum of a lighting and sanitizing device according to the prior art;

FIG. 9 is a graph showing the trend of the energy of the photons as the wavelength increases;

FIGS. 10 and 11 are schematic views of lighting apparatuses that integrate the lighting and sanitizing device according to the invention.

We must clarify that in the present description the phraseology and terminology used, as well as the figures in the attached drawings also as described, have the sole function of better illustrating and explaining the present invention, their function being to provide a non-limiting example of the invention itself, since the scope of protection is defined by the claims

To facilitate comprehension, the same reference numbers have been used, where possible, to identify identical common elements in the drawings. It is understood that elements and characteristics of one embodiment can be conveniently combined or incorporated into other embodiments without further clarifications.

### DESCRIPTION OF SOME EMBODIMENTS OF THE PRESENT INVENTION

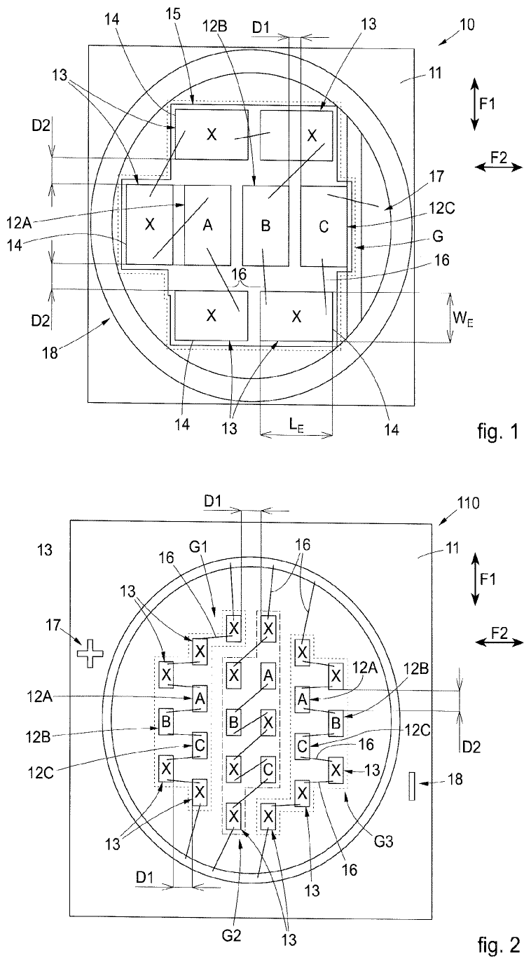

With reference to FIGS. 1-4, a lighting and sanitizing device 10 according to the present invention is described, which can be integrated in lighting apparatuses 20, for example bulbs of the type illustrated in FIG. 10 or 11, or in spotlights, lamps, light strips, and the like, with the function of illuminating and at the same time breaking down the microbial load in a surrounding environment, both on surfaces and of the air.

The lighting and sanitizing device 10 comprises an electronic board 11 on which there is installed at least one electromagnetic radiation emitter group G comprising a triad of primary emitters 12 and five secondary emitters 13.

The electronic board 11 may be a substrate where at least one emitter group G is installed.

The electronic board 11 can be a support package, for example a PCB, where at least one emitter group G is installed.

According to the embodiment in FIG. 1 a single emitter group G is provided, while in the embodiments of FIGS. 2-4 there are illustrated examples comprising respectively 3, 6 and 12 emitter groups G1-G12.

The number of emitter groups G may vary indicatively between 2 and 20, for example two, three, five, eight, fifteen, seventeen, etc. although it may also be greater as a function of the overall dimensions of the lighting and sanitizing device 10.

According to the invention, the or each emitter group G comprises a triad of primary emitters 12, indicated as 12A, 12B, 12C, each configured to emit an electromagnetic radiation with wavelength centred on a specific peak value, different from each other, and five secondary emitters 13 configured to emit an electromagnetic radiation with a wavelength greater than that of the primary emitters 12A, 12B, 12C.

Preferably each emitter group G consists of eight emitters, of which a triad of primary emitters 12 and five secondary emitters 13.

In the figures, in order to simplify the identification thereof, the primary emitters 12A, 12B, 12C are indicated with the respective letter “A”, “B”, “C”, while the secondary emitters are indicated with the letter “X”.

Preferably all the secondary emitters 13 are configured to emit an electromagnetic radiation having the same wavelength.

The triad of primary emitters 12A, 12B, 12C comprises:
a first emitter 12A configured to emit an electromagnetic radiation having a peak value with a first wavelength λ1 comprised between 404 nm and 410 nm, with a preferential peak value of 407 nm;
a second emitter 12B configured to emit an electromagnetic radiation having a peak value with a second wavelength λ2 comprised between 410 nm and 416 nm, with a preferential peak value of 413 nm;
a third emitter 12C configured to emit an electromagnetic radiation having a peak value with a third wavelength λ3 comprised between 418 nm and 424 nm, with a preferential peak value of 421 nm.

The five secondary emitters 13 are configured to emit an electromagnetic radiation having a peak value with a fourth wavelength λ4 comprised between 452 nm and 458 nm, with a preferential peak value of 455 nm.

Other components, not illustrated, such as for example one or more temperature sensing sensors, power supply, conversion systems, microcontrollers or the like may also be provided on the electronic board 11.

According to a further aspect of the invention, the primary 12 and secondary 13 emitters comprise a chip 14, in the sector also commonly called DIE, made of semiconductor material and configured to emit a substantially mono-frequency highly focused radiation with the desired wavelength. For example, this may be achieved by doping the semiconductor material.

Preferably, both the primary emitters 12A, 12B, 12C and the secondary emitters 13 are configured to emit a radiation within a very narrow and precise range of the wavelength, for example of about 4-8 nm centred on the respective peak value. Preferably, the range of the wavelength around the respective peak value is ±3 nm.

In the case where there are N emitter groups G, G1-G12, each comprising eight emitters 12A, 12B, 12C, 13, in total in the lighting and sanitizing device 10 there are N first emitters 12A, N second emitters 12B, N third emitters 12C and 5*N secondary emitters 13 in such a way that the proportion 12A:12B:12C:13=1:1:1:5 is always maintained.

According to embodiments, the electronic board 11, or at least the portion thereof on which the emitters 12, 13 are installed, has a substantially circular, or rectangular, or square shape depending on the uses, so as to allow the emission of an overall light ray within a well-defined cone.

In accordance with embodiments, when there are at least two emitter groups G as in FIGS. 2-4, the emitters 12, 13 of one and of the other group G are substantially homogeneously distributed on the surface of the electronic board 11.

Preferably the primary emitters 12A, 12B, 12C are more concentrated in a central zone or band, and are at least partly surrounded by the secondary emitters 13, so as to obtain a better emission uniformity.

According to embodiments, the emitters 12, 13 are preferably arranged aligned in a same direction, or part on a first direction F1 and part on a second direction F2 transverse to the first direction F1.

In accordance with embodiments, at least the primary emitters 12A, 12B, 12C are all oriented in the same direction (FIGS. 1-4). In the case of a plurality of groups G, all primary 12 and secondary 13 emitters can all be oriented in the same direction.

According to embodiments, the emitters 12, 13 are positioned such that in at least one direction F1, F2 first emitters 12A, second emitters 12B and third emitters 12C, i.e. primary emitters 12 having the same wavelength/frequency are not directly side by side.

In particular, in the embodiments of FIGS. 1-4 in the first direction indicated by arrow F1, i.e. in the direction parallel to a longitudinal axis of most emitters 12, 13, two identical first emitters 12A, second emitters 12B, or third emitters 12C are never provided adjacent to each other.

According to one aspect of the invention, the chips 14 of the primary 12 and/or secondary 13 emitters have homogeneous dimensions and shapes, for example rectangular, square, circular or other.

According to embodiments, the chips 14 of all emitters 12, 13 have substantially the same shape and dimension.

According to embodiments, the primary 12 and secondary 13 emitters all have a circular conformation.

According to embodiments, the primary emitters 12 and the secondary emitters 13 all have a rectangular conformation, which may have a ratio between a long side and a short side comprised between 1.5 and 1.6.

According to embodiments, each chip 14 has a short side comprised between 0.5 and 0.6 mm and a long side comprised between 0.8 and 0.9 mm.

In the embodiment of FIG. 1 the first 12A, second 12B and third 12C primary emitters and a secondary emitter 13 are arranged with respective longitudinal axes parallel to the first direction F1, while the remaining four secondary emitters 13 are arranged along the second direction F2, on the one side and on the other side in such a way as to substantially homogeneously occupy a circular surface of the electronic board 11.

In the embodiment of FIG. 2, wherein the lighting and sanitizing device 110 comprises three emitter groups G1, G2, G3 the primary emitters 12 and the secondary emitters 13 are distributed on rows aligned on parallel axes in the first direction F1. The emitter groups G1, G2, G3 can de facto be positioned one after the other in the second transverse direction F2.

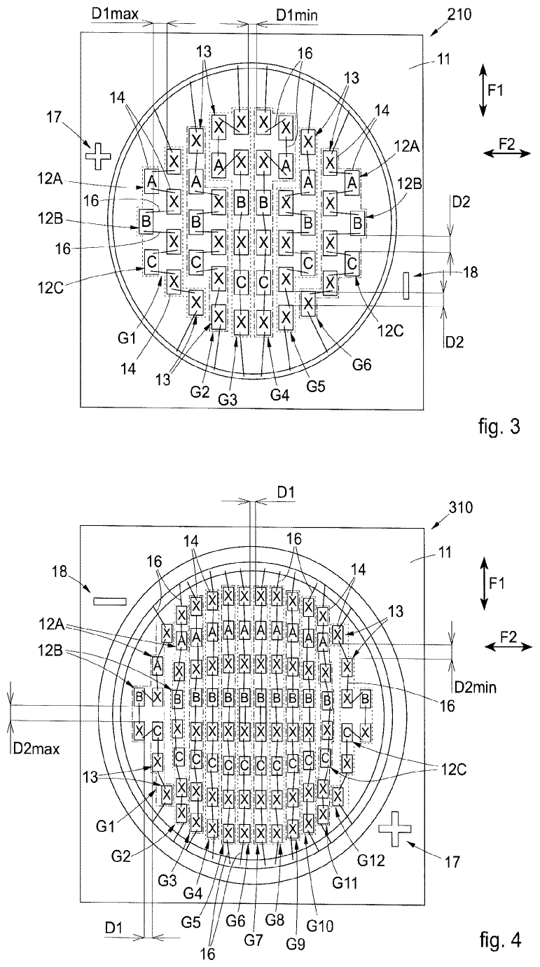

FIG. 3 shows a lighting and sanitizing device 210 comprising six emitter groups G1-G6, wherein the primary emitters 12 and the secondary emitters 13 are distributed in rows aligned along axes parallel with respect to the first direction F1, and the groups G1-G6 are positioned substantially one after the other in the second direction F2.

FIG. 4 shows a lighting and sanitizing device 310 comprising twelve emitter groups G1-G12, wherein the primary emitters 12 and the secondary emitters 13 are distributed in rows aligned along axes at least partly parallel to each other.

It can be noted that in this embodiment the first emitters 12A, the second emitters 12B and the third emitters 12C are mostly aligned on transverse rows extending in the second direction F2 with respect to the respective longitudinal axes aligned in the first direction F1 according to a substantially matrix scheme.

Between one row and the other of the primary emitters 12A, 12B and 12B, 12C there is provided a row of secondary emitters 13.

In accordance with embodiments, the emitters 12, 13 are connected by gold connecting wires 16, preferably gold having a purity greater than 99%, more preferably greater than 99.95%.

Preferably, the emitter groups G, G1-G12 are connected in parallel to each other between a respective positive pole 17 and a negative pole 18 of an electrical circuit.

This solution allows the proportion between the emitters 1:1:1:5 described above to be kept constant even in the event of a malfunction of a given group G, G1-G12.

According to embodiments, the distance D1, D2 between the chips 14 of the side-by-side emitters 12, 13 is comprised between 0.001 mm and 1.1 mm, preferably between 0.1 and 0.9 mm.

According to embodiments, the “horizontal” distance D1 between adjacent chips 14 in the second direction F2 may be equal to or different from the “vertical” distance D2 in the first direction F1.

For example, in the embodiment of FIG. 1, the “horizontal” distance D1 can be about half of the “vertical” distance D2.

By way of example, the distance D1 may be about 0.15 mm, while the distance D2 may be about 0.28 mm.

In the embodiment of FIG. 2, the distances D1 and D2 may be substantially the same or very similar to each other. By way of example, the distance D1 may be about 0.70 mm and the distance D2 about 0.71 mm.

By way of example, the distance D1 may be about 0.15 mm, while the distance D2 may be about 0.28 mm.

In the embodiment of FIG. 3, the distance D1 may vary between an emitter 12, 13 and the one adjacent in the direction transverse to the longitudinal axes, for example between a minimum value D1min of about 0.3 mm and a maximum value of about 0.5 mm, while the distance D2 in the direction parallel to the longitudinal axes may be substantially constant at around 0.5 mm.

In the embodiment of FIG. 3, the distance D1 may vary between an emitter 12, 13 and the one adjacent in the direction transverse to the longitudinal axes, for example between a minimum value D1min of about 0.3 mm and a maximum value of about 0.48 mm, while the distance D2 in the direction parallel to the longitudinal axes may vary between a minimum value D2min of about 0.80 mm to a maximum value of 1.02 mm.

It is clear, however, that the distances between the different primary emitters 12 and/or secondary emitters 13 on the electronic board 11 is determined both by the dimension of the electronic board 11 itself, and by the dimension, shape and number of the primary emitters 12 and secondary emitters 13 installed thereon.

According to embodiments, the chips 14 are made of semiconductor material based on indium gallium nitride (InGaN), each having an indium to gallium ratio suitable for emitting the electromagnetic radiation with the desired peak wavelength.

According to embodiments, the lighting and sanitizing device 10 comprises a photosensitive coating layer 15 arranged so as to enclose the at least one emitter group G, G1-G12 and configured to modify the overall electromagnetic spectrum emitted by the at least one emitter group G, G1-G12.

According to embodiments, the photosensitive coating layer 15 is configured to emit, in combination with the respective primary emitters 12 and secondary emitters 13, a spectrum with wavelength comprised between 400 nm and 700 nm, in which the peak values of the wavelengths λ1, λ2, λ3 of the primary emitters 12 and possibly also the peak value of the wavelength λ4 of the secondary emitters 13 are present.

According to embodiments, the photosensitive coating layer 15 is configured to modify the frequency, i.e. the wavelength of the emitted radiation, bringing it at least partially outside the specific frequency/wavelength of the respective emitter group G, G1-G12, but letting pass, substantially without modifying them, i.e. without significantly affecting them, the radiations with wavelengths λ1, λ2, λ3 emitted by the primary emitters 12, so as to have a high sanitizing effect on the sensitive microorganisms in this band.

According to embodiments, the photosensitive coating layer 15 can be based on phosphorus, and be suitably doped with substances suitable for obtaining a spectrum of white light. For example, the photosensitive coating layer 15 may be partially doped with substances adapted to provide a red colour, so as to compensate for the blue colour.

This allows to obtain a white light emission centred on the planckian curve having a high colour rendering index (CRI), preferably higher than 80 and even more preferably higher than 90 and therefore proximate to natural light, and consequently a controlled and precise energy emission is obtained.

Note that the photosensitive coating layer 15 may be provided in all embodiments described and illustrated in the accompanying figures, regardless of the number of emitter groups G.

According to embodiments, there is a single photosensitive coating layer 15 that commonly covers all the emitter groups G of the lighting and sanitizing device 10, i.e. the entire area occupied by them.

According to possible variants, it may be provided that, in the event that several emitter groups G are present, each of them is provided with a respective photosensitive coating layer 15 that covers all the emitters 12, 13 belonging to a specific emitter group G. In this case, the photosensitive coating layers 15 associated with one or the other emitter group G are preferably substantially equal to each other.

According to further variants, it may also be provided that each of the primary emitters 12 and secondary emitters 13 is provided with its own photosensitive coating layer 15, wherein the various photosensitive coating layers 15 are preferably substantially equal to each other.

According to a possible variant not illustrated, the photosensitive coating layer 15 can be applied only on some of the individual emitters 12, 13 of the or of each emitter group G, preferably discretely and differentiated in relation to the type of emitter 12, 13, i.e. to the specific wavelength of the emitted radiation.

According to embodiments, in addition to the photosensitive coating layer 15, it may be provided for further photosensitive coating layers superimposed on each other, which may be the same or different as a function of the effect it is wished to be obtained.

The emitters 12, 13 are preferably of the high light efficiency type, preferably comprised between 120 and 200 lm/w, even more preferably comprised between 140 and 180 lm/w.

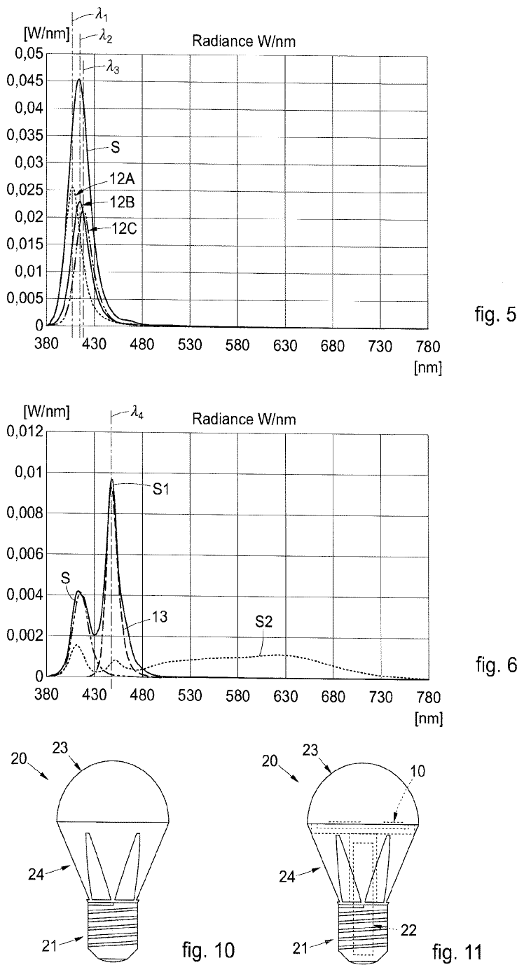

FIGS. 5-7 illustrate the spectra of the electromagnetic radiation emitted respectively by the triad of primary emitters 12, by one or more emitter groups G comprising the triad of primary emitters 12 and the five secondary emitters 13, or respective multiples, and by one or more emitter groups G in combination with a photosensitive coating layer 15.

As can be seen in FIG. 5, the presence of the three electromagnetic radiations having respective wavelengths λ1, λ2, λ3 centred on peak values close to each other allows to obtain a resulting spectral emission S that covers substantially the entire Soret band, so as to be strongly effective on certain microorganisms.

FIG. 6 shows the radiations emitted respectively by the triad of primary emitters 12 and by the secondary emitters 13, the spectral emission of the emitters S1 given by the combination of the radiations of the primary 12 and secondary 13 emitters and the resulting spectral emission S2 given by the combination of the radiations of the primary 12 and secondary 13 emitters and of the photosensitive coating layer 15.

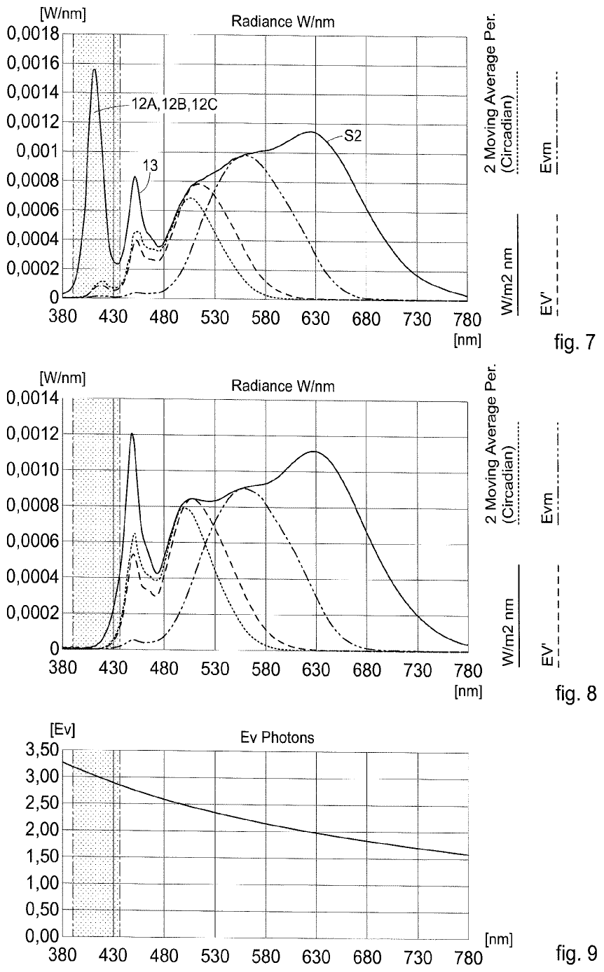

FIG. 7, finally, shows the resulting spectral emission S2 with a different scale than that of FIG. 6, so as to be able to observe the overall trend thereof in detail.

The peak value at 455 nm of the secondary emitters 13 was found to be an optimal value to excite the phosphor present in the photosensitive coating layer 15 and appropriately compensate for the spectral radiation of the lighting and sanitizing device 10, to bring back the colour rendering values (for example indicated as colour rendering index (CRI or TM-30) on the planckian curve, and optimize the overall energy emission.

As can be noted from the graph in FIG. 6, the photosensitive coating layer 15 has a high influence on the radiations emitted by the secondary emitters 13, i.e. on the wavelengths around 450 nm and 460 nm, at which a break-down in radiance by around 90% is found, since the energy is transformed to obtain a continuous resulting spectral emission S2 with white light. The reduction in radiance, on the other hand, is much less at the wavelengths between 400 nm and 420 nm, between 60% and 65%.

Consequently, the resulting spectral emission S2 of the lighting and sanitizing device 10 according to the invention has a first peak value centred in the Soret band, a second peak value centred around the peak value of the secondary emitters 13 and a bell-shaped trend with a further peak at around the wavelengths between 620 nm and 640 nm.

The configuration of the lighting and sanitizing device 10 of the invention, thanks to the proportion between the various types of primary emitters 12 and secondary emitters 13, provides better sanitizing performance than a known device comprising three blue-violet type LED emitters separated from each other and a white LED emitter.

FIG. 8 illustrates the resulting spectral emission of this known device. As can be noted, in this case the spectral emission does not have any peak value around the Soret band, so it is not very effective against microorganisms sensitive to this band, and long exposure times are necessary to obtain a certain level of sanitation.

The lighting and sanitizing device 10 according to the invention allows to obtain, with the same geometry of the lighting apparatus 20 in which it is inserted, and with the same power absorbed by the lighting and sanitizing device 10 with respect to the known device, an evident reduction in break-down times necessary to obtain a certain level of reduction of the microorganisms.

This is also achieved thanks to the fact that the energy of the photons is higher at low wavelengths and tends to decrease as the wavelengths grow. Obtaining a spectral emission having a peak value at relatively low frequencies within the Soret band therefore allows to obtain high-energy photons that allow to interfere better with the microorganisms and in shorter time.

Below are four tables with relative examples, in which the characteristics and the results obtained with the known device comprising the three separate blue-violet LEDs are compared with those obtained with a lighting and sanitizing device 10 according to the invention.

The examples reported below are related to bacteria (a group of species typically present under real conditions) and viruses (SARS-COVID2), but they can also be extended to other microorganisms.

For each example, the minimum energy doses are reported, expressed in Joules/cm² delivered by each of the devices considered to break down viruses and bacteria respectively in order to obtain a break-down level, expressed as a logarithmic reduction, respectively equal to 90% (1−Log) and 99% (2−Log).

| | Known | Present |
|---|---|---|
| Quantities | device | invention (FIG. 4) |
| Electrical consumption | 51 | |
| of the complete | | |
| apparatus (W) | | |
| Luminous flux (lm) | 4354 | 4356 |
| Light beam aperture | 34° | |
| Colour rendering (CRI) | >92 | |
| Colour temperature (° K) | 4020 | 4089 |
| Minimum germicidal | 0.72 | |
| dose 1-Log | | |
| (J/cm2) - Bacteria | | |
| Time for 1-Log reduction | 00 d:0 1 h:04 m:35 s = | 00 d:00 h:39 m:24 s = |
| (1 metre) - Bacteria | 64 m:35 s | 39 m:24 s |
| Minimum germicidal | 0.79 | |
| dose 2-Log | | |
| (J/cm2) - Bacteria | | |
| Time for 2-Log reduction | 00 d:01 h:11 m:04 s = | 00 d:00 h:43 m:22 s = |
| (1 metre) - Bacteria | 71 m:04 s | 43 m:22 s |
| Minimum germicidal | 0.76 | |
| dose 1-Log | | |
| (J/cm2) - Virus | | |
| Time for 1-Log reduction | 00 d:01 h:08 m:40 s = | 00 d:00 h:41 m:54 s = |
| (1 metre) - Virus | 68 m:40 s | 41 m:54 s |
| Minimum germicidal | 1.86 | |
| dose 2-Log | | |
| (J/cm2) - Virus | | |
| Time for 2-Log reduction | 00 d:02 h:47 m:51 s = | 00 d:01 h:42 m:24 s = |
| (1 metre) - Virus | 167 m:51 s | 102 m:24 s |

| | Previous | Present |
|---|---|---|
| Quantities | version | invention (FIG. 3) |
| Electrical consumption | 24 | |
| of the complete | | |
| apparatus (W) | | |
| Luminous flux (lm) | 1663 | 1666 |
| Light beam aperture | 75° | |
| Colour rendering (CRI) | >92 | |
| Colour temperature (° K) | 4020 | 4089 |
| Minimum germicidal | 0.72 | |
| dose 1-Log | | |
| (J/cm2) - Bacteria | | |
| Time for 1-Log reduction | 00 d:12 h:28 m:02 s = | 00 d:07 h:35 m:49 s = |
| (1 metre) - Bacteria | 748 m:02 s | 455 m:49 s |
| Minimum germicidal | 0.79 | |
| dose 2-Log | | |
| (J/cm2) - Bacteria | | |
| Time for 2-Log reduction | 00 d:13 h:43 m:09 s = | 00 d:08 h:21 m:35 s = |
| (1 metre) - Bacteria | 823 m:09 s | 521 m:35 s |
| Minimum germicidal | 0.76 | |
| dose 1-Log | | |
| (J/cm2) - Virus | | |
| Time for 1-Log reduction | 00 d:13 h:15 m:19 s = | 00 d:08 h:04 m:38 s = |
| (1 metre) - Virus | 795 m:19 s | 484 m:38 s |
| Minimum germicidal | 1.86 | |
| dose 2-Log | | |
| (J/cm2) - Virus | | |
| Time for 2-Log reduction | 01 d:08 h:23 m:52 s = | 00 d:19 h:44 m:30 s = |
| (1 metre) - Virus | 1943 m:52 s | 1184 m:30 s |

| | Previous | Present |
|---|---|---|
| Quantities | version | invention (FIG. 1) |
| Electrical consumption | 45 | |
| of the complete | | |
| apparatus (W) | | |
| Luminous flux (lm) | 5503 | 3851 |
| Light beam aperture | 106° | |
| Colour rendering (CRI) | >92 | |
| Colour temperature (° K) | 4020 | 3759 |
| Minimum germicidal | 0.72 | |
| dose 1-Log | | |
| (J/cm2) - Bacteria | | |
| Time for 1-Log reduction | 00 d:09 h:09 m:49 s = | 00 d:05 h:43 m:16 s = |
| (1 metre) - Bacteria | 549 m:49 s | 343 m:16 s |
| Minimum germicidal | 0.79 | |
| dose 2-Log | | |
| (J/cm2) - Bacteria | | |
| Time for 2-Log reduction | 00 d:10 h:05 m:02 s = | 00 d:06 h:17 m:44 s = |
| (1 metre) - Bacteria | 605 m:02 s | 202 m:44 s |
| Minimum germicidal | 0.76 | |
| dose 1-Log | | |
| (J/cm2) - Virus | | |
| Time for 1-Log reduction | 00 d:09 h:44 m:35 s = | 00 d:06 h:04 m:58 s = |
| (1 metre) - Virus | 584 m:35 s | 364 m:58 s |
| Minimum germicidal | 1.86 | |
| dose 2-Log | | |
| (J/cm2) - Virus | | |
| Time for 2-Log reduction | 00 d:23 h:48 m:48 s = | 00 d:14 h:52 m:01 s = |
| (1 metre) - Virus | 1428 m:48 s | 892 m:01 s |

| | Previous | Present |
|---|---|---|
| Quantities | version | invention (FIG. 1) |
| Electrical consumption | 27 | |
| of the complete | | |
| apparatus (W) | | |
| Luminous flux (lm) | 3658 | 4052 |
| Light beam aperture | 123° | 86.6° |
| Colour rendering (CRI) | >92 | |
| Colour temperature (° K) | 4020 | 3973 |
| Minimum germicidal | 0.72 | |
| dose 1-Log | | |
| (J/cm2) - Bacteria | | |
| Time for 1-Log reduction | 00 d:11 h:39 m:10 s = | 00 d:02 h:35 m:09 s = |
| (1 metre) - Bacteria | 699 m:10 s | 155 m:09 s |
| Minimum germicidal | 0.79 | |
| dose 2-Log | | |
| (J/cm2) - Bacteria | | |
| Time for 2-Log reduction | 00 d:12 h:49 m:02 s = | 00 d:02 h:50 m:44 s = |
| (1 metre) - Bacteria | 769 m:02 s | 170 m:44 s |
| Minimum germicidal | 0.76 | |
| dose 1-Log | | |
| (J/cm2) - Virus | | |
| Time for 1-Log reduction | 00 d:12 h:26 m:40 s = | 00 d:02 h:44 m:58 s = |
| (1 metre) - Virus | 746 m:40 s | 164 m:58 s |
| Minimum germicidal | 1.86 | |
| dose 2-Log | | |
| (J/cm2) - Virus | | |
| Time for 2-Log reduction | 01 d:06 h:24 m:59 s = | 00 d:06 h:43 m:12 s = |
| (1 metre) - Virus | 1824 m:59 s | 403 m:12 s |

As can be seen from the tables reported here, the lighting and sanitizing device 10 of the present invention allows to reduce the times at least by 35-40% compared to the known device with the same doses delivered and aperture angle of the light beam.

Furthermore, in the case where a lighting apparatus having a narrower light beam aperture is used as in Example 4, the times are further reduced, reaching an overall reduction by 77-78%.

Note that the colour temperature difference in example 4 falls within the tolerance ranges defined by the ANSI standard for lighting categories (about +/−150K) whereby the sources can be considered substantially of the same colour dimming. It should also be noted that the removal times of the device according to the invention are shorter than those of the known device regardless of whether the luminous flux is greater (example 4), lower (example 3) or substantially equal (examples 1 and 2).

According to embodiments, each emitter 12, 13 has dimensions such that a width thereof W_{E} has a ratio with a length thereof L_{E} such that the relation between them is expressed with an index R_{E}=W_{E}/L_{E} comprised between 0 and 1, with zero excluded.

In particular, such measurements W_{E}, L_{E} may correspond respectively to the width and length of each chip 14 (or DIE) of an emitter 12, 13 (FIG. 1).

In the case of emitters 12, 13 of circular or square shape, the values of width W_{E} and length L_{E} can be substantially equal, while in the case of emitters 12, 13 of rectangular shape the index R_{E} is comprised between 0 and 1, extremes excluded.

Furthermore, between each of the emitters 12, 13, i.e. between the respective chips 14 there is a minimum distance D_{E} not lower than 1 m (D_{E}≥1 m), i.e. 0.001 mm. The exemplary distances D1, D2, D1min, D2min, D1max, D2max indicated in the attached figures are therefore in any case greater than or equal to D_{E}.

In particular, the lighting and sanitizing device 10 has emitters 12, 13 with dimensions such that they are spaced from each other in such a way as to ensure a homogeneous and uniform distribution of the electromagnetic radiation over the incident space.

According to possible embodiments, at least one lighting and sanitizing device 10 may be included in a lighting apparatus 20. For example, the lighting apparatus 20 may comprise one or more lighting and sanitizing devices 10, 110, 210, 310 according to any of the possible embodiments.

According to possible exemplary embodiments, the lighting apparatus 20 may comprise a projector with COB, or other systems that provide for the use of COB where the latter may comprise one or more lighting and sanitizing devices 10, 110, 210, 310.

According to further exemplary embodiments, the lighting apparatus 20 may comprise a panel on which one or more lighting and sanitizing devices 10, 110, 210, 310, also of a different type, are installed. In this exemplary case, the lighting apparatus 20 may comprise one or more light diffusers and/or a high reflectance panel adapted to recover light emitted in directions other than that desired.

FIGS. 10 and 11 illustrate a lighting apparatus 20 comprising a connection fitting 21, which may be of a conventional and standardized type. The lighting apparatus 20 comprises a power supply 22, a cap 23 for emitting the light cone and a heatsink 24.

A lighting and sanitizing device 10 according to the invention is arranged inside the cap 23.

It is clear that modifications and/or additions of parts may be made to the lighting and sanitizing device 10 as described heretofore, without departing from the field and scope of the present invention, as defined by the claims.

It is also clear that, although the present invention has been described with reference to some specific examples, a person of skill in the art shall certainly be able to achieve other equivalent forms of the lighting and sanitizing device having the characteristics as set forth in the claims and hence all coming within the field of protection defined thereby.

In the following claims, the sole purpose of the references in brackets is to facilitate their reading and they must not be considered as restrictive factors with regard to the field of protection defined by the claims.

1. A lighting and sanitizing device comprising an electronic board comprising at least one electromagnetic radiation emitter group wherein said at least one emitter group comprises a triad of primary emitters and five secondary emitters, wherein said triad of primary emitters comprises:
a first emitter configured to emit an electromagnetic radiation having a peak value with wavelength (λ1) comprised between 404 nm and 410 nm;
a second emitter configured to emit an electromagnetic radiation having a peak value with wavelength (λ2) comprised between 410 nm and 416 nm;
a third emitter configured to emit an electromagnetic radiation having a peak value with wavelength (λ3) comprised between 418 nm and 424 nm;
and wherein said five secondary emitters are configured to emit an electromagnetic radiation having a peak value with a wavelength (λ4) comprised between 452 nm and 458 nm.

2. The lighting and sanitizing device according to claim 1, comprising a photosensitive coating layer arranged to enclose said at least one emitter group and configured to modify the overall electromagnetic spectrum emitted by said at least one emitter group to emit a visible electromagnetic spectrum comprised between 400 nm and 700 nm in which the peak values of said primary emitters are present.

3. The lighting and sanitizing device according to claim 1, comprising a number N of emitter groups comprised between 2 and 20, wherein there are N first emitters, N second emitters, N third emitters and 5*N secondary emitters.

4. The lighting and sanitizing device according to claim 1, wherein the emitters of said at least one emitter group are distributed on the surface of said electronic board so that the overlapping volume of the respective emitted light cones comprises the light cones of the primary emitters and at least one of the light cones of the secondary emitters.

5. The lighting and sanitizing device according to claim 1, wherein said primary emitters are positioned on said electronic board along at least a first direction, wherein along said at least a first direction there are no first emitters adjacent to each other, second emitters adjacent to each other and third emitters adjacent to each other.

6. The lighting and sanitizing device according to claim 1, wherein a distance between primary emitters and/or first close secondary emitters is comprised between 0.001 mm and 1.10 mm, preferably between 0.1 and 0.9 mm.

7. The lighting and sanitizing device according to claim 1, wherein said primary emitters are more concentrated in a central zone or band of said electronic board and are at least partly surrounded by said secondary emitters.

8. The lighting and sanitizing device according to claim 1, wherein said primary emitters and/or said secondary emitters are LED emitters.

9. The lighting and sanitizing device according to claim 1, wherein each of said primary emitters and each of said secondary emitters has a substantially circular shape.

10. The lighting apparatus comprising one or more lighting and sanitizing devices according to claim 1.
